(19) United States
(12) Patent Application Publication
(10) Pub. No.: US 2022/0362528 A1
(43) Pub. Date: Nov. 17, 2022

(54) Scalp Treatment Applicator Assembly

(71) Applicant: Rodney Williams, Forth Worth, TX (US)

(72) Inventor: Rodney Williams, Forth Worth, TX (US)

(21) Appl. No.: 17/317,584

(22) Filed: May 11, 2021

(51) Int. Cl. A61M 35/00

(52) U.S. Cl. CPC A61M 35/003; A61M 2202/04; A61M 2205/075; A61M 2210/06; A61M 2210/04

(57) ABSTRACT

A scalp treatment applicator assembly includes a tube which contains a fluid scalp treatment. A cone is fluidly coupled to the tube and the cone is in fluid communication with an interior of the tube to receive the fluid scalp treatment. The cone tapers to point to penetrate the user's hair to apply the fluid scalp treatment to the user's scalp without disturbing the user's hair. A bulb is removably attachable to the tube and the bulb is comprised of a resiliently deformable material such that the bulb can be compressed by the user. The bulb is in fluid communication with an interior of the tube when the bulb is attached to the tube to urge the fluid scalp treatment outwardly through the cone when the bulb is compressed.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to applicator devices and more particularly pertains to a new applicator device for applying a scalp treatment directly to a user's scalp. The device includes a tube that contains a fluid scalp treatment and a cone that is attached to the tube for applying the fluid treatment directly to the user's scalp. The device also includes a bulb, comprised of a deformable material that is removably attached to the tube for urging the fluid scalp treatment outwardly through the cone.

### (2) Description of Related Art Including Information Disclosed Under 37 CFR 1.97 and 1.98

The prior art relates to applicator devices including a variety of syringe devices that include a plunger for applying various cosmetic solutions to a user. The prior art discloses a tube which contains a cosmetic solution and an application tip that is attachable to a cone which is disposed on the tube. In no instance does the prior art disclose a tube which contains a fluid solution, a cone that is attached to the tube for dispensing the fluid solution and a bulb that is removably attachable to the tube for urging the fluid solution outwardly through the cone.

### BRIEF SUMMARY OF THE INVENTION

An embodiment of the disclosure meets the needs presented above by generally comprising a tube which contains a fluid scalp treatment. A cone is fluidly coupled to the tube and the cone is in fluid communication with an interior of the tube to receive the fluid scalp treatment. The cone tapers to point to penetrate the user's hair to apply the fluid scalp treatment to the user's scalp without disturbing the user's hair. A bulb is removably attachable to the tube and the bulb is comprised of a resiliently deformable material such that the bulb can be compressed by the user. The bulb is in fluid communication with an interior of the tube when the bulb is attached to the tube to urge the fluid scalp treatment outwardly through the cone when the bulb is compressed.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a scalp treatment applicator assembly according to an embodiment of the disclosure.

FIG. 2 is an exploded perspective view of an embodiment of the disclosure.

FIG. 3 is a front view of an embodiment of the disclosure.

FIG. 4 is a left side view of an embodiment of the disclosure.

FIG. 5 is a perspective in-use view of an embodiment of the disclosure.

### DETAILED DESCRIPTION OF THE INVENTION

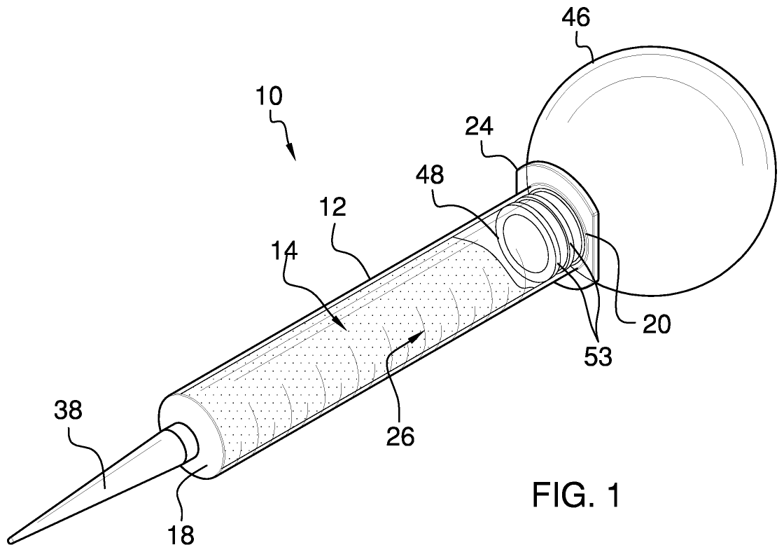

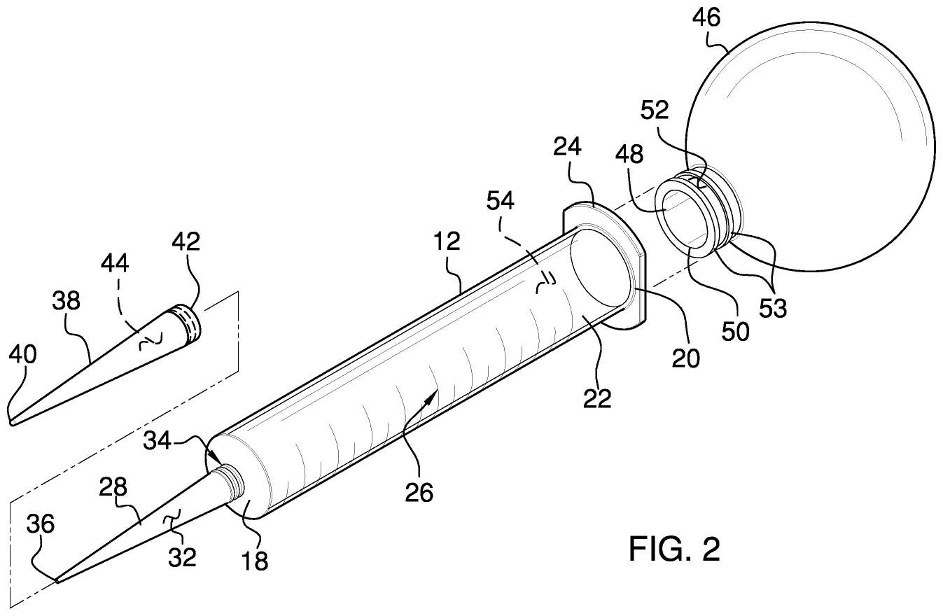

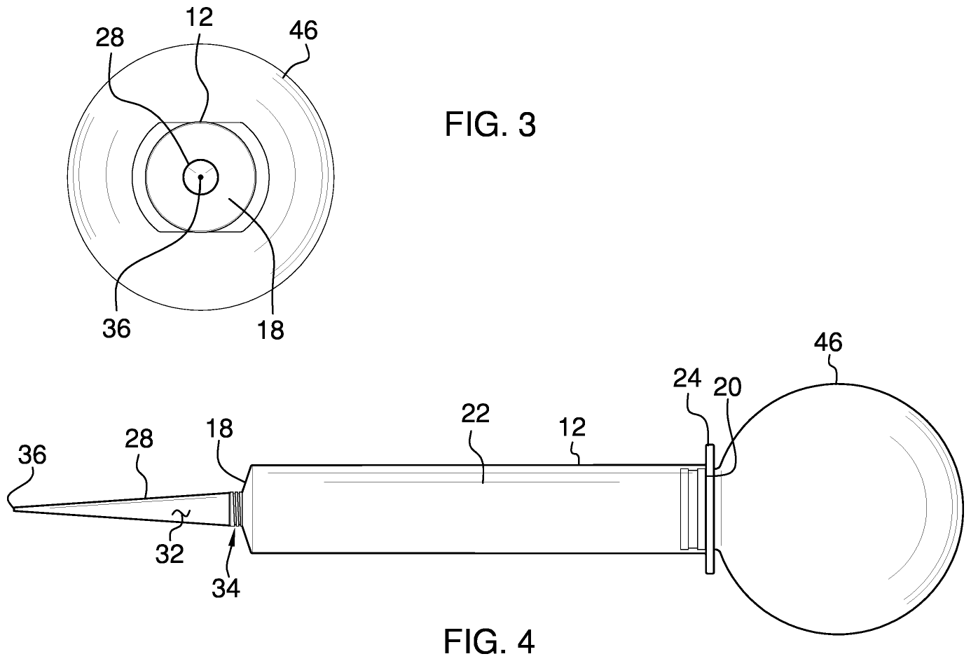

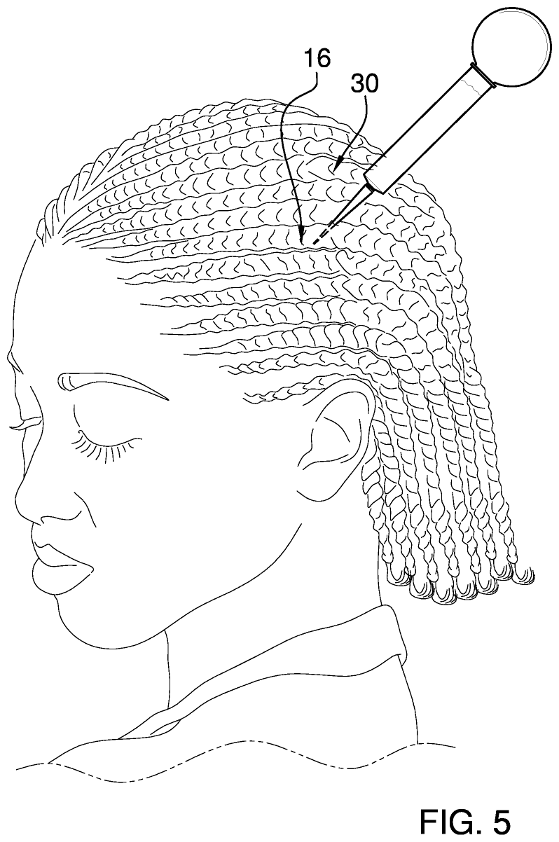

With reference now to the drawings, and in particular to FIGS. 1 through 5 thereof, a new applicator device embodying the principles and concepts of an embodiment of the disclosure and generally designated by the reference numeral 10 will be described.

As best illustrated in FIGS. 1 through 5, the scalp treatment applicator assembly 10 generally comprises a tube 12 that contains a fluid scalp treatment 14. The fluid scalp treatment 14 may be any solution that is applied to a user's scalp 16 for treating psoriasis, dandruff or any other scalp condition that would require the application of a fluid treatment. The tube 12 has a first end 18, a second end 20 and an outer wall 22 extending between the first end 18 and the second end 20. The second end 20 is open into an interior of the tube 12 to facilitate the tube 12 to be filled with the fluid scalp treatment 14. The outer wall 22 has a lip 24 extending outwardly therefrom, the lip 24 extends around a full circumference of the outer wall 22 and the lip 24 is aligned with the second end 20. Indicia 26 are printed on the outer wall 22 and the indicia 26 comprise a graduated scale to indicate the fluid volume of the fluid scalp treatment 14 in the tube 12.

A cone 28 is fluidly coupled to the tube 12 and the cone 28 is in fluid communication with an interior of the tube 12 to receive the fluid scalp treatment 14. The cone 28 tapers to point to penetrate the user's hair 30 for applying the fluid scalp treatment 14 to the user's scalp 16 without disturbing the user's hair 30. The cone 28 is positioned on the first end 18 of the tube 12, the cone 28 has an outer surface 32 and the outer surface 32 has a threaded portion 34 that is positioned adjacent to the first end 18 of the tube 12. The cone 28 has a distal end 36 with respect to the first end 18 and the distal end 36 is open to dispense the fluid scalp treatment 14.

A conical cap 38 is positionable over the cone 28 for closing the cone 28 to inhibit the fluid scalp treatment 14 from escaping through the cone 28. The conical cap 38 has a primary end 40, a secondary end 42 and an inner surface 44, and the conical cap 38 tapers to a point at the primary end 40. The secondary end 42 insertably receives the distal end 36 of the cone 28 when the conical cap 38 is positioned on the cone 28. Moreover, the inner surface 44 is threaded adjacent to the secondary end 42 thereby facilitating the inner surface 44 to threadably engage the threaded portion 34 of the outer surface of the cone 28.

A bulb 46 is provided that is removably attachable to the tube 12, and the bulb 46 is comprised of a resiliently deformable material, such as rubber or other type of fluid impermeable material. In this way the bulb 46 can be compressed by the user while inhibiting the fluid scalp treatment 14 from passing through the bulb 46. The bulb 46 is in fluid communication with an interior of the tube 12 when the bulb 46 is attached to the tube 12. In this way the bulb 46 can urge the fluid scalp treatment 14 outwardly through the cone 28 when the bulb 46 is compressed.

The bulb 46 has a stem 48 extending away from the bulb 46 and the stem 48 has a distal end 50 with respect to the bulb 46 and an outer surface 52. The distal end 50 of the stem 48 is open to pass air into the cylinder when the bulb 46 is compressed. Additionally, the outer surface of the stem 48 has a series of rings 53 each extending outwardly from the outer surface 52 of the stem 48. The rings 53 are spaced apart from each other and are distributed between the distal end 50 of the stem 48 and the bulb 46. Each of the rings 53 frictionally engages an interior surface 54 of the outer wall 22 of the tube 12 when the stem 48 is inserted into the second end 20 of the cylinder for retaining the stem 48 in the cylinder.

In use, the tube 12 is filled with the pre-determined amount of the fluid scalp treatment 14 and the stem 48 on the bulb 46 is inserted into the second end 20 of the tube 12. The cone 28 is extended through the user's hair 30 such that the distal end 36 of the cone 28 is positioned adjacent to the user's scalp 16. The bulb 46 is compressed to urge the fluid scalp treatment 14 onto the user's scalp 16. In this way the fluid scalp treatment 14 can be applied to the user's scalp 16 without disturbing the user's hair 30, thereby preserving braids, cornrows or other hair 30 styles that the user has. The conical cap 38 is positioned on the cone 28 to close the cone 28 and retain the fluid scalp treatment 14 in the tube 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A scalp treatment applicator assembly for applying a liquid treatment to a scalp without disturbing a user's hairstyle, said assembly comprising:
a tube containing a fluid scalp treatment;
a cone being fluidly coupled to said tube, said cone being in fluid communication with an interior of said tube wherein said cone is configured to receive the fluid scalp treatment, said cone tapering to point wherein said cone is configured to penetrate the user's hair to apply the fluid scalp treatment to the user's scalp without disturbing the user's hair;
a conical cap being positionable over said cone for closing said cone wherein said conical cap is configured to inhibit the fluid scalp treatment from escaping through said cone; and
a bulb being removably attachable to said tube, said bulb being comprised of a resiliently deformable material wherein said bulb is configured to be compressed by the user, said bulb being in fluid communication with an interior of said tube when said bulb is attached to said tube wherein said bulb is configured to urge the fluid scalp treatment outwardly through said cone when said bulb is compressed.

2. The assembly according to claim 1, wherein said tube has a first end, a second end and an outer wall extending between said first end and said second end, said second end being open into an interior of said tube wherein said second end is configured to facilitate said tube to be filled with the fluid scalp treatment, said outer wall having a lip extending outwardly therefrom, said lip extending around a full circumference of said outer wall, said lip being aligned with said second end, said outer wall having indicia being printed thereon, said indicia comprising a graduated scale wherein said graduated scale is configured to indicate the fluid volume of the fluid scalp treatment in said tube.

3. The assembly according to claim 2, wherein said cone is positioned on said first end of said tube, said cone having an outer surface, said outer surface having a threaded portion being positioned adjacent to said first end of said tube, said cone having a distal end with respect to said first end, said distal end being open wherein said distal end is configured to dispense the fluid scalp treatment.

4. The assembly according to claim 3, wherein said conical cap has a primary end, a secondary end and an inner surface, said conical cap tapering to a point at said primary end, said secondary end insertably receiving said distal end of said cone when said conical cap is positioned on said cone, said inner surface being threaded adjacent to said secondary end thereby facilitating said inner surface to threadably engage said threaded portion of said outer surface of said cone.

5. A scalp treatment applicator assembly for applying a liquid treatment to a scalp without disturbing a user's hairstyle, said assembly comprising:
a tube being configured to contain a fluid scalp treatment, said tube having a first end, a second end and an outer wall extending between said first end and said second end, said second end being open into an interior of said tube wherein said second end is configured to facilitate said tube to be filled with the fluid scalp treatment, said outer wall having a lip extending outwardly therefrom, said lip extending around a full circumference of said outer wall, said lip being aligned with said second end, said outer wall having indicia being printed thereon, said indicia comprising a graduated scale wherein said graduated scale is configured to indicate the fluid volume of the fluid scalp treatment in said tube;
a cone being fluidly coupled to said tube, said cone being in fluid communication with an interior of said tube wherein said cone is configured to receive the fluid scalp treatment, said cone tapering to point wherein said cone is configured to penetrate the user's hair to apply the fluid scalp treatment to the user's scalp without disturbing the user's hair, said cone being positioned on said first end of said tube, said cone having an outer surface, said outer surface having a threaded portion being positioned adjacent to said first end of said tube, said cone having a distal end with respect to said first end, said distal end being open wherein said distal end is configured to dispense the fluid scalp treatment;
a conical cap being positionable over said cone for closing said cone wherein said conical cap is configured to inhibit the fluid scalp treatment from escaping through said cone, said conical cap having a primary end, a secondary end and an inner surface, said conical cap tapering to a point at said primary end, said secondary end insertably receiving said distal end of said cone when said conical cap is positioned on said cone, said inner surface being threaded adjacent to said secondary end thereby facilitating said inner surface to threadably engage said threaded portion of said outer surface of said cone; and
a bulb being removably attachable to said tube, said bulb being comprised of a resiliently deformable material wherein said bulb is configured to be compressed by the user, said bulb being in fluid communication with an interior of said tube when said bulb is attached to said tube wherein said bulb is configured to urge the fluid scalp treatment outwardly through said cone when said bulb is compressed, said bulb having a stem extending away from said bulb, said stem having a distal end with respect to said bulb and an outer surface, said distal end of said stem being open wherein said distal end of said stem is configured to pass air into said cylinder when said bulb is compressed, said outer surface of said stem having a series of rings each extending outwardly from said outer surface of said stem, said rings being spaced apart from each other and being distributed between said distal end of said stem and said bulb, each of said rings frictionally engaging an interior surface of said outer wall of said tube when said stem is inserted into said second end of said cylinder for retaining said stem in said cylinder.

6. A method of applying a scalp treatment to a user's scalp without disturbing the user's hair, the steps of the method comprising:
providing a tube having a first end and a second end, said second end being open to facilitate said tube to be filled with a fluid scalp treatment;
providing a cone being fluidly coupled to said tube such that said cone is in fluid communication with an interior of said tube thereby facilitating said cone to receive the fluid scalp treatment, said cone having a distal end being open for dispensing the fluid scalp treatment outwardly through said distal end;
providing a conical cap;
providing a bulb being removably attachable to said tube, said bulb being comprised of a resiliently deformable material thereby facilitating said bulb to be compressed by a user;
filling said tube with a pre-determined volume of the fluid scalp treatment;
attaching said bulb to said second end of said tube;
extending said cone through the user's hair such that said distal end of said cone is positioned adjacent to the user's scalp;
compressing said bulb to urge the fluid scalp treatment onto the user's scalp; and
positioning said conical cap on said cone for closing said cone thereby inhibiting the fluid scalp treatment from escaping said cone.
